(19) United States
(12) Patent Application Publication
(10) Pub. No.: US 2024/0245714 A1
(43) Pub. Date: Jul. 25, 2024

(54) COMPOSITION AND METHODS USING COMBINATION OF NARH AND NR TO PRODUCE INTRACELLULAR NICOTINAMIDE ADENINE DINUCLEOTIDE (NAD+)

(71) Applicant: SOCIETE DES PRODUITS NESTLE S.A., Vevey (CH)

(72) Inventors: CARLES CANTO ALVAREZ, Cuarnens (CH); ELEONORA CIARLO, Lausanne (CH); MARIA PILAR GINER, Lausanne (CH); SOFIA MOCO, Amsterdam (NL)

(21) Appl. No.: 18/558,808

(22) PCT Filed: Apr. 25, 2022
(86) PCT No.: PCT/EP2022/060882

§ 371 Date: Nov. 3, 2023

(30) Foreign Application Priority Data
May 5, 2021 (EP) 21172203.8

(51) Int. Cl. A61K 31/706; A61K 9/00

(52) U.S. Cl. CPC A61K 31/706; A61K 9/0053

(57) ABSTRACT

Compositions and methods that use a combination of NARH and NR to produce intracellular NAD+/NADH. The compositions can be used in food or beverage applications, pharmaceutical formulations, or as a dietary supplement. The compositions can be administered to an individual to promote the increase of intracellular levels of nicotinamide adenine dinucleotide (“NAD+”) in cells and tissues for improving cell and tissue survival or overall cell and tissue health. Also, the compositions can be administered to an individual to treat or prevent a mitochondria-related disease or a condition associated with altered mitochondrial function in an individual in need thereof or at risk thereof. The mitochondria-related disease or condition is selected from the group consisting of deleterious effects of aging, stress (e.g., oxidative stress), obesity, overweight, reduced metabolic rate, metabolic syndrome, diabetes mellitus, complications from diabetes, hyperlipidemia, neurodegenerative disease, cognitive disorder, stress-induced or stress-related cognitive dysfunction, mood disorder, anxiety disorder, age-related neuronal death or dysfunction, chronic kidney disease, kidney failure, trauma, infection, cancer, hearing loss, macular degeneration, myopathies and dystrophies, and combinations thereof.

### FIELD OF THE INVENTION

The present disclosure generally relates to compositions and methods that use a combination of NARH and NR to produce intracellular NAD+/NADH. Intracellular levels of NAD+ can be increased in cells and tissues to improve cell and tissue survival and/or or overall cell and tissue health.

### BACKGROUND OF THE INVENTION

Nicotinic acid and nicotinamide are the vitamin forms of nicotinamide adenine dinucleotide (NAD+). Eukaryotes can synthesize NAD+de novo via the kynurenine pathway from tryptophan, and niacin supplementation prevents the pellagra that can occur in populations with a tryptophan-poor diet. Nicotinic acid is phosphoribosylated to nicotinic acid mononucleotide (NaMN), which is then adenylylated to form nicotinic acid adenine dinucleotide (NaAD), which in turn is amidated to form NAD+.

NAD+ is an enzyme co-factor that is essential for the function of several enzymes related to reduction-oxidation reactions and energy metabolism. NAD+ functions as an electron carrier in cell metabolism of amino acids, fatty acids, and carbohydrates. NAD+ serves as an activator and substrate for sirtuins, a family of protein deacetylases that have been implicated in metabolic function and extended lifespan in lower organisms. The co-enzymatic activity of NAD+, together with the tight regulation of its biosynthesis and bioavailability, makes it an important metabolic monitoring system that is clearly involved in the aging process.

### SUMMARY OF THE INVENTION

The present disclosure provides a composition comprising a combination of NARH and NR or consisting of a combination of NARH and NR for use in a method for increasing intracellular nicotinamide adenine dinucleotide (NAD+) in an individual, the method comprising orally administering to the subject a composition comprising a combination of NARH and NR or consisting of a combination of NARH and NR in an amount effective to increase NAD+ biosynthesis in one or more cells of the individual.

The increase in NAD+ biosynthesis can provide one or more benefits to the individual, for example a human (e.g., a human undergoing medical treatment), a pet or a horse (e.g., a pet or horse undergoing medical treatment), or cattle or poultry (e.g., cattle or poultry being used in agriculture). The one or more benefits can comprise at least one of increased mitochondrial energy, treatment or prevention of metabolic fatigue, treatment or prevention of muscle fatigue, improvement in a physiological state linked to metabolic fatigue in one or more cells, improved mobility or improved longevity. Preferably, the NAD+ biosynthesis is increased in one or more cells of the individual, for example one or more cells that are part of at least one body part selected from the group consisting of a liver, a kidney, a brain, and a skeletal muscle.

In an embodiment, the composition is administered enterally.

In an embodiment, the composition is selected from the group consisting of a food product, a food supplement, an oral nutritional supplement (ONS), a medical food, and combinations thereof.

In another embodiment, the present disclosure provides a unit dosage form of a composition comprising a combination of NARH and NR or consisting of a combination of NARH and NR, in an amount effective to increase NAD+ biosynthesis in an individual. The composition can be selected from the group consisting of a food product, a food supplement, an oral nutritional supplement (ONS), a medical food, and combinations thereof.

In another embodiment, the present disclosure provides a method of achieving at least one result selected from the group consisting of (i) increased mitochondrial energy in one or more cells, (ii) improvement in a physiological state linked to metabolic fatigue in one or more cells, (iii) treatment or prevention of metabolic fatigue in one or more cells, (iv) treatment or prevention of muscle fatigue, (v) improved mobility and (vii) improved longevity. Preferably, at least a portion of the one or more cells are part of at least one body part selected from the group consisting of a liver, a kidney, a brain, and a skeletal muscle. The method comprises orally administering to an individual a composition comprising a combination of NaRH and NR in an amount effective to increase NAD+ biosynthesis.

In another embodiment, the present disclosure provides a method of treating or preventing (e.g., reducing incidence and/or severity) a mitochondria-related disease or a condition associated with altered mitochondrial function in an individual in need thereof or at risk thereof. The method comprises orally administering to an individual a composition comprising a combination of NARH and NR in an amount effective to increase NAD+ biosynthesis.

The mitochondria-related disease or condition can be selected from the group consisting of deleterious effects of aging, stress (e.g., oxidative stress), obesity, overweight, reduced metabolic rate, metabolic syndrome, diabetes mellitus, complications from diabetes, hyperlipidemia, neurodegenerative disease, cognitive disorder, stress-induced or stress-related cognitive dysfunction, mood disorder, anxiety disorder, age-related neuronal death or dysfunction, premature aging syndromes (progeria, cockayne syndrome), chronic kidney disease, kidney failure, trauma, infection, cancer, hearing loss, macular degeneration, myopathies and dystrophies, mitochondrial genetic diseases and combinations thereof.

In another embodiment, the present disclosure provides a method of promoting protective immunity and/or for preventing and/or treating bacterial or viral infections and/or for limiting immune mediated pathology following infection in an individual comprising delivering to the individual in need thereof or at risk thereof, the method comprising orally administering to the individual a composition combination of NARH and NR or consisting of a combination of NARH and NR in an amount effective to increase NAD+ biosynthesis.

In another embodiment, the present disclosure provides a unit dosage form of a composition comprising a combination of NARH and NR or consisting of a combination of NARH and NR in an amount effective to treat or prevent (e.g., reducing incidence and/or severity) a mitochondria-related disease or a condition associated with altered mitochondrial function in an individual in need thereof or at risk thereof. The composition can be selected from the group consisting of a food product, a food supplement, an oral nutritional supplement (ONS), a medical food, and combinations thereof.

An advantage of one or more embodiments provided by the present disclosure is to potentiate benefits on oxidative metabolism and prevent DNA damage.

Another advantage of one or more embodiments provided by the present disclosure is to replenish NAD+ pools, which decline with age.

Yet another advantage of one or more embodiments provided by the present disclosure is to help off-set slowing of the metabolism associated with aging.

Another advantage of one or more embodiments provided by the present disclosure is to help increase fatty acids metabolism.

Yet another advantage of one or more embodiments provided by the present disclosure is to help the body to metabolize fat and increase lean body mass.

Another advantage of one or more embodiments provided by the present disclosure is to help maintain heart health.

Yet another advantage of one or more embodiments provided by the present disclosure is to help support healthy LDL-cholesterol and fatty acid levels in the blood.

Additional features and advantages are described in, and will be apparent from, the following Detailed Description and the Figures.

### BRIEF DESCRIPTION OF THE DRAWINGS

FIG. 1. NARH and NR combination synergistically induces NAD+ in mouse hepatocytes.

AML12 cells were treated with dihydronicotinic acid riboside (NARH) and nicotinamide riboside (NR), alone or in combination for 2 hours. NARH and NR were used at the concentration of 0.5 mM. After treatment, cells were trypsinized and collected for NAD+ and protein content measurements. NAD+ was extracted and quantified using an EnzyChrom NAD/NADH Assay Kit (BioAssay Systems; #E2ND-100) according to the manufacturer's instructions. Proteins were quantified with BCA Protein Assay Kit (Pierce™; #23225). All results are expressed as mean+/−SD of n=3-4.

FIG. 2. NARH and NA do not exhibit synergism in NAD+ induction.

AML12 cells were treated with dihydronicotinic acid riboside (NARH) and nicotinic acid (NA), alone or in combination for 2 hours. NARH and NA were used at the concentration of 0.5 mM. After treatment, cells were trypsinized and collected for NAD+ and protein content measurements. NAD+ was extracted and quantified using an EnzyChrom NAD/NADH Assay Kit (BioAssay Systems; #E2ND-100) according to the manufacturer's instructions. Proteins were quantified with BCA Protein Assay Kit (Pierce™; #23225). All results are expressed as mean+/−SD of n=2.

FIG. 3. NARH and NAM do not exhibit synergism in NAD+ induction.

AML12 cells were treated with dihydronicotinic acid riboside (NARH) and nicotinamide (NAM) alone or in combination for 2 hours. NARH and NAM were used at the concentration of 0.5 and 5 mM, respectively. After treatment, cells were trypsinized and collected for NAD+ and protein content measurements. NAD+ was extracted and quantified using an EnzyChrom NAD/NADH Assay Kit (BioAssay Systems; #E2ND-100) according to the manufacturer's instructions. Proteins were quantified with BCA Protein Assay Kit (Pierce™; #23225). All results are expressed as mean+/−SD of n=2.

FIG. 4. NARH and NAR do not exhibit synergism in NAD+ induction.

AML12 cells were treated with dihydronicotinic acid riboside (NARH) and nicotinic acid riboside (NAR) alone or in combination for 2 hours. NARH and NAR were used at the concentration of 0.5 mM. After treatment, cells were trypsinized and collected for NAD+ and protein content measurements. NAD+ was extracted and quantified using an EnzyChrom NAD/NADH Assay Kit (BioAssay Systems; #E2ND-100) according to the manufacturer's instructions. Proteins were quantified with BCA Protein Assay Kit (Pierce™; #23225). All results are expressed as mean+/−SD of n=2.

FIG. 5. NARH and NRH do not exhibit synergism in NAD+ induction.

AML12 cells were treated with dihydronicotinic acid riboside (NARH) and dihydronicotinamide riboside (NRH) alone or in combination for 2 hours. NARH and NRH were used at the concentration of 0.5 and 0.01 mM, respectively. After treatment, cells were trypsinized and collected for NAD+ and protein content measurements. NAD+ was extracted and quantified using an EnzyChrom NAD/NADH Assay Kit (BioAssay Systems; #E2ND-100) according to the manufacturer's instructions. Proteins were quantified with BCA Protein Assay Kit (Pierce™; #23225). All results are expressed as mean+/−SD of n=4.

FIG. 6. NR and NA do not exhibit synergism in NAD+ induction.

AML12 cells were treated with nicotinamide riboside (NR) and nicotinic acid (NA) alone or in combination for 2 hours. NR and NA were used at the concentration of 0.5 mM. After treatment, cells were trypsinized and collected for NAD+ and protein content measurements. NAD+ was extracted and quantified using an EnzyChrom NAD/NADH Assay Kit (BioAssay Systems; #E2ND-100) according to the manufacturer's instructions. Proteins were quantified with BCA Protein Assay Kit (Pierce™; #23225). All results are expressed as mean+/−SD of n=2.

FIG. 7. NR and NAM do not exhibit synergism in NAD+ induction.

AML12 cells were treated with nicotinamide riboside (NR) and nicotinamide (NAM) alone or in combination for 2 hours. NR and NAM were used at the concentration of 0.5 and 5 mM respectively. After treatment, cells were trypsinized and collected for NAD+ and protein content measurements. NAD+ was extracted and quantified using an EnzyChrom NAD/NADH Assay Kit (BioAssay Systems; #E2ND-100) according to the manufacturer's instructions. Proteins were quantified with BCA Protein Assay Kit (Pierce™; #23225). All results are expressed as mean+/−SD of n=2.

FIG. 8. NR and NAR do not exhibit synergism in NAD+ induction.

AML12 cells were treated with nicotinamide riboside (NR) and nicotinic acid riboside (NAR) alone or in combination for 2 hours. NR and NAR were used at the concentration of 0.5 mM. After treatment, cells were trypsinized and collected for NAD+ and protein content measurements. NAD+ was extracted and quantified using an EnzyChrom NAD/NADH Assay Kit (BioAssay Systems; #E2ND-100) according to the manufacturer's instructions. Proteins were quantified with BCA Protein Assay Kit (Pierce™; #23225). All results are expressed as mean+/−SD of n=2.

FIG. 9. NA and NAM do not exhibit synergism in NAD+ induction.

AML12 cells were treated with nicotinic acid (NA) and nicotinamide (NAM) alone or in combination for 2 hours. NA and NAM were used at the concentration of 0.5 and 5 mM, respectively. After treatment, cells were trypsinized and collected for NAD+ and protein content measurements. NAD+ was extracted and quantified using an EnzyChrom NAD/NADH Assay Kit (BioAssay Systems; #E2ND-100) according to the manufacturer's instructions. Proteins were quantified with BCA Protein Assay Kit (Pierce™; #23225). All results are expressed as mean+/−SD of n=2.

FIG. 10. NA and NAR do not exhibit synergism in NAD+ induction.

AML12 cells were treated with nicotinic acid (NA) and nicotinic acid riboside (NAR) alone or in combination for 2 hours. NA and NAR were used at the concentration of 0.5 mM. After treatment, cells were trypsinized and collected for NAD+ and protein content measurements. NAD+ was extracted and quantified using an EnzyChrom NAD/NADH Assay Kit (BioAssay Systems; #E2ND-100) according to the manufacturer's instructions. Proteins were quantified with BCA Protein Assay Kit (Pierce™; #23225). All results are expressed as mean+/−SD of n=2.

FIG. 11. NAM and NAR do not exhibit synergism in NAD+ induction.

AML12 cells were treated with nicotinamide (NAM), nicotinic acid riboside (NAR) alone or in combination for 2 hours. NAM and NAR were used at the concentration of 5 and 0.5 mM, respectively. After treatment, cells were trypsinized and collected for NAD+ and protein content measurements. NAD+ was extracted and quantified using an EnzyChrom NAD/NADH Assay Kit (BioAssay Systems; #E2ND-100) according to the manufacturer's instructions. Proteins were quantified with BCA Protein Assay Kit (Pierce™; #23225). All results are expressed as mean+/−SD of n=2.

FIG. 12. NAR and NRH do not exhibit synergism in NAD+ induction.

AML12 cells were treated with nicotinic acid riboside (NAR), dihydronicotinamide riboside (NRH) alone or in combination for 2 hours. NAR and NRH were used at the concentration of 0.5 and 0.01 mM, respectively. After treatment, cells were trypsinized and collected for NAD+ and protein content measurements. NAD+ was extracted and quantified using an EnzyChrom NAD/NADH Assay Kit (BioAssay Systems; #E2ND-100) according to the manufacturer's instructions. Proteins were quantified with BCA Protein Assay Kit (Pierce™; #23225). All results are expressed as mean+/−SD of n=4.

FIG. 13. NARH and NR combination synergistically induces NAD+ in rat pancreatic p cells.

INS-1 cells were treated with dihydronicotinic acid riboside (NARH) and nicotinamide riboside (NR), alone or in combination for 2 hours. NARH and NR were used at the concentration of 0.5 mM. After treatment, cells were trypsinized and collected for NAD+ and protein content measurements. NAD+ was extracted and quantified using an EnzyChrom NAD/NADH Assay Kit (BioAssay Systems; #E2ND-100) according to the manufacturer's instructions. Proteins were quantified with BCA Protein Assay Kit (Pierce™; #23225). All results are expressed as mean+/−SD of n=2.

FIG. 14. NARH and NR combination, but not NARH or NR alone, induces NAD+ in mouse fibroblast.

MEF cells were treated with dihydronicotinic acid riboside (NARH) and nicotinamide riboside (NR), alone or in combination for 2 hours. NARH and NR were used at the concentration of 0.5 mM. After treatment, cells were trypsinized and collected for NAD+ and protein content measurements. NAD+ was extracted and quantified using an EnzyChrom NAD/NADH Assay Kit (BioAssay Systems; #E2ND-100) according to the manufacturer's instructions. Proteins were quantified with BCA Protein Assay Kit (Pierce™; #23225). All results are expressed as mean+/−SD of n=2. NARH vs control P=0.7581; NR vs control P=0.5337; NRH+NR vs control P=0.0002.

FIG. 15. NARH and NR combination, but not NARH or NR alone, induces NAD+ in mouse macrophages.

RAW 264.7 cells were treated with dihydronicotinic acid riboside (NARH) and nicotinamide riboside (NR), alone or in combination for 2 hours. NARH and NR were used at the concentration of 0.5 mM. After treatment, cells were scraped and collected for NAD+ and protein content measurements. NAD+ was extracted and quantified using an EnzyChrom NAD/NADH Assay Kit (BioAssay Systems; #E2ND-100) according to the manufacturer's instructions. Proteins were quantified with BCA Protein Assay Kit (Pierce™; #23225). All results are expressed as mean+/−SD of n=2-4. NARH vs control P=0.8130; NR vs control P=0.0788; NRH+NR vs control P=0.0087.

FIG. 16. NR and NARH have synergistic effects on hepatic NAD⁺ levels.

After a 2 hour fast, mice (n=5 per group) were intraperitoneally injected with saline (as vehicle) or 500 mg/kg of NR, NARH or NR+NARH. For the NR+NARH combination, in one group both compounds were injected using a single mixture with both compounds (NR+NARH Comb) and in the second group the compounds were injected separately, one in the left side and one in the right side of the peritoneum (NR+NARH Sep). One hour later, liver tissue was snap frozen and NAD⁺ levels were measured. All data is presented as mean+/−SEM for n=4-5 mice per group. * indicates p<0.05 vs. vehicle treated group. # indicates p<0.05 vs. single compound treatments.

FIG. 17. The combination of NR+NARH leads to increased intracellular content of NRH in AML12 hepatocytes.

AML12 cells were treated with either PBS (as control), NR (0.5 mM), NARH (0.5 mM) or both. Then, 2 hours later, cells were flash-frozen and processed to evaluate intracellular levels through LC-MS methods (see Giner et al., 2021; doi: 10.3390/ijms221910598). All data is presented as mean+/−SEM of n=3 experiments.

FIG. 18. NR and NARH lead to increased NRH levels in circulation.

After a 2 hour fast, mice (n=5 per group) were intraperitoneally injected with saline (as vehicle) or 500 mg/kg of NR, NARH or NR+NARH. For the NR+NARH combination, in one group both compounds were injected using a single mixture with both compounds (NR+NARH Comb) and a second group where the compounds were injected separately, one in the left and one in the right side of the peritoneum (NR+NARH Sep). One hour later, blood was collected and NRH levels were measured by LC-MS (see Giner et al., 2021; doi: 10.3390/ijms221910598). All data is presented as mean+/−SEM of n=3 experiments.

FIG. 19. NR and NARH require the NRH path for NAD⁺ synthesis, characterized by adenosine kinase activity.

AML12 cells were treated with DMSO (as vehicle) or the adenosine kinase inhibitor 5-IT (1 μM;) for 1 hour and then treated with either PBS (as control), NR (0.5 mM), NARH (0.5 mM) or both. Two hours later, acidic extracts were obtained to evaluate NAD⁺ levels. All data is presented as mean+/−SEM of n=3 experiments. * indicates p<0.05 vs. the respective vehicle treated group.

### DETAILED DESCRIPTION OF THE INVENTION

### Definitions

Some definitions are provided hereafter. Nevertheless, definitions may be located in the “Embodiments” section below, and the above header “Definitions” does not mean that such disclosures in the “Embodiments” section are not definitions.

All percentages expressed herein are by weight of the total weight of the composition unless expressed otherwise. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number.

All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in this disclosure and the appended claims, the singular forms “a,” “an” and “the” include plural referents unless the context clearly dictates otherwise. Thus, for example, reference to “a component” or “the component” includes two or more components.

The words “comprise,” “comprises” and “comprising” are to be interpreted inclusively rather than exclusively. Likewise, the terms “include,” “including” and “or” should all be construed to be inclusive, unless such a construction is clearly prohibited from the context. Nevertheless, the compositions disclosed herein may lack any element that is not specifically disclosed herein.

Thus, a disclosure of an embodiment using the term “comprising” includes a disclosure of embodiments “consisting essentially of” and “consisting of” the components identified. Any embodiment disclosed herein can be combined with any other embodiment disclosed herein.

The term “and/or” used in the context of “X and/or Y” should be interpreted as “X,” or “Y,” or “X and Y.” Similarly, “at least one of X or Y” should be interpreted as “X,” or “Y,” or “X and Y.” For example, “at least one of metabolic fatigue or muscle fatigue” should be interpreted as “metabolic fatigue,” or “muscle fatigue,” or “both metabolic fatigue and muscle fatigue.”

Where used herein, the terms “example” and “such as,” particularly when followed by a listing of terms, are merely exemplary and illustrative and should not be deemed to be exclusive or comprehensive. As used herein, a condition “associated with” or “linked with” another condition means the conditions occur concurrently, preferably means that the conditions are caused by the same underlying condition, and most preferably means that one of the identified conditions is caused by the other identified condition.

The terms “food,” “food product” and “food composition” mean a product or composition that is intended for ingestion by an individual such as a human and provides at least one nutrient to the individual. A food product typically includes at least one of a protein, a lipid, a carbohydrate and optionally includes one or more vitamins and minerals. The compositions of the present disclosure, including the many embodiments described herein, can comprise, consist of, or consist essentially of the elements disclosed herein, as well as any additional or optional ingredients, components, or elements described herein or otherwise useful in a diet.

As used herein, the term “isolated” means removed from one or more other compounds or components with which the compound may otherwise be found, for example as found in nature.

For example, “isolated” preferably means that the identified compound is separated from at least a portion of the cellular material with which it is typically found in nature. In an embodiment, an isolated compound is free from any other compound.

“Prevention” includes reduction of risk, incidence and/or severity of a condition or disorder. The terms “treatment,” “treat” and “to alleviate” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The term does not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment,” “treat” and “to alleviate” are also intended to include the potentiation or otherwise enhancement of one or more primary prophylactic or therapeutic measure. The terms “treatment,” “treat” and “to alleviate” are further intended to include the dietary management of a disease or condition or the dietary management for prophylaxis or prevention a disease or condition. A treatment can be patient- or doctor-related.

The term “unit dosage form,” as used herein, refers to physically discrete units suitable as unitary dosages for human and animal subjects, each unit containing a predetermined quantity of the composition disclosed herein in an amount sufficient to produce the desired effect, in association with a pharmaceutically acceptable diluent, carrier or vehicle. The specifications for the unit dosage form depend on the particular compounds employed, the effect to be achieved, and the pharmacodynamics associated with each compound in the host.

The term “combination”, or terms “in combination”, “used in combination with” or “combined preparation” as used herein may refer to the combined administration of two or more agents simultaneously, sequentially or separately.

The term “simultaneous” as used herein means that the agents are administered concurrently, i.e. at the same time.

The term “sequential” as used herein means that the agents are administered one after the other.

The term “separate” as used herein means that the agents are administered independently of each other but within a time interval that allows the agents to show a combined, preferably synergistic, effect. Thus, administration “separately” may permit one agent to be administered, for example, within 1 minute, 5 minutes or 10 minutes after the other.

As used herein, an “effective amount” is an amount that prevents a deficiency, treats a disease or medical condition in an individual, or, more generally, reduces symptoms, manages progression of the disease, or provides a nutritional, physiological, or medical benefit to the individual. The relative terms “improve,” “increase,” “enhance,” “promote” and the like refer to the effects of the composition disclosed herein, namely a composition comprising a combination of NaRH and NR, relative to a composition not having a combination of NaRH and NR but otherwise identical. As used herein, “promoting” refers to enhancing or inducing relative to the level before administration of the composition disclosed herein.

A “subject” or “individual” is a mammal, preferably a human. The term “elderly” in the context of a human means an age from birth of at least 60 years, preferably above 63 years, more preferably above 65 years, and most preferably above 70 years. The term “older adult” in the context of a human means an age from birth of at least 45 years, preferably above 50 years, more preferably above 55 years, and includes elderly individuals.

“Mobility” is the ability to move independently and safely from one place to another.

“Metabolic fatigue” means reduced mitochondrial function in one or more cells (e.g., one or more of liver, kidney, brain, skeletal muscle) due to a shortage of substrates within the one or more cells and/or due to an accumulation of metabolites within the one or more cells which interfere with mitochondrial function.

“Overweight” is defined for a human as a body mass index (BMI) between 25 and 30 kg/m2. “Obese” is defined for a human as a BMI of at least 30 kg/m2, for example 30-39.9 kg/m2. “Weight loss” is a reduction of the total body weight. Weight loss may, for example, refer to the loss of total body mass in an effort to improve one or more of health, fitness or appearance.

“Diabetes” encompasses both the type I and type II forms of the disease. Non-limiting examples of risk factors for diabetes include: waistline of more than 40 inches for men or 35 inches for women, blood pressure of 130/85 mmHg or higher, triglycerides above 150 mg/dl, fasting blood glucose greater than 100 mg/dl or high-density lipoprotein of less than 40 mg/dl in men or 50 mg/dl in women.

As used herein, the term “metabolic syndrome” refers to a combination of medical disorders that, when occurring together, increase the risk of developing cardiovascular disease and diabetes. It affects one in five people in the United States and prevalence increases with age. Some studies have shown the prevalence in the United States to be an estimated 25% of the population. In accordance with the International Diabetes Foundation consensus worldwide definition (2006), metabolic syndrome is central obesity plus any two of the following:
Raised triglycerides: >150 mg/dL (1.7 mmol/L), or specific treatment for this lipid abnormality;
Reduced HDL cholesterol: <40 mg/dL (1.03 mmol/L) in males, <50 mg/dL (1.29 mmol/L) in females, or specific treatment for this lipid abnormality;
Raised blood pressure: systolic BP>130 or diastolic BP>85 mm Hg, or treatment of previously diagnosed hypertension; and
Raised fasting plasma glucose: (FPG)>100 mg/dL (5.6 mmol/L), or previously diagnosed type 2 diabetes.

As used herein, “neurodegenerative disease” or “neurodegenerative disorder” refers to any condition involving progressive loss of functional neurons in the central nervous system. In an embodiment, the neurodegenerative disease is associated with age-related cell death. Non-limiting examples of neurodegenerative diseases include mild cognitive impairment, Alzheimer's disease, Parkinson's disease, Huntington's disease, amyotrophic lateral sclerosis (also known as ALS and as Lou Gehrig's disease), peripheral neuropathy, AIDS dementia complex, adrenoleukodystrophy, Alexander disease, Alper's disease, ataxia telangiectasia, Batten disease, bovine spongiform encephalopathy (BSE), Canavan disease, corticobasal degeneration, Creutzfeldt-Jakob disease, dementia with Lewy bodies, fatal familial insomnia, frontotemporal lobar degeneration, Kennedy's disease, Krabbe disease, Lyme disease, Machado-Joseph disease, multiple sclerosis, multiple system atrophy, neuroacanthocytosis, Niemann-Pick disease, Pick's disease, primary lateral sclerosis, progressive supranuclear palsy, Refsum disease, Sandhoff disease, diffuse myelinoclastic sclerosis, spinocerebellar ataxia, subacute combined degeneration of spinal cord, tabes dorsalis, Tay-Sachs disease, toxic encephalopathy, transmissible spongiform encephalopathy, and wobbly hedgehog syndrome. The present disclosure is not limited to a specific embodiment of the neurodegenerative disease, and the neurodegenerative disease can be any neurologically-related condition known to one skilled in this art.

As used herein, “cognitive function” refers to any mental process that involves symbolic operations, e.g., perception, memory, attention, speech comprehension, speech generation, reading comprehension, creation of imagery, learning, and reasoning, preferably at least memory.

Methods for measuring cognitive function are well-known and can include, for example, individual or battery tests for any aspect of cognitive function. One such test is the Prudhoe Cognitive Function Test by Margallo-Lana et al. (2003) J. Intellect. Disability Res. 47:488-492. Another such test is the Mini Mental State Exam (MMSE), which is designed to assess orientation to time and place, registration, attention and calculation, recall, language use and comprehension, repetition, and complex commands. Folstein et al. (1975) J. Psych. Res. 12:189-198. Such tests can be used to assess cognitive function in an objective manner, so that changes in cognitive function, for example in response to treatment in accordance with methods disclosed herein, can be measured and compared.

As used herein, a “cognitive disorder” refers to any condition that impairs cognitive function. Non-limiting examples of a cognitive disorder include delirium, dementia, learning disorder, attention deficit disorder (ADD), and attention deficit hyperactivity disorder (ADHD).

As used herein, NARH is reduced form of nicotinic acid riboside (NAR) and NR relates to nicotinamide riboside.

### Embodiments

The present disclosure provides a composition comprising a combination of NARH and NR or consisting of a combination of NARH and NR for use in a method for increasing intracellular nicotinamide adenine dinucleotide (NAD+) in an individual, the method comprising orally administering to the subject a composition comprising a combination of NARH and NR or consisting of a combination of NARH and NR in an amount effective to increase NAD+ biosynthesis in one or more cells of the individual.

The increase in NAD+ biosynthesis can provide one or more benefits to the individual, for example a human (e.g., a human undergoing medical treatment), a pet or a horse (e.g., a pet or horse undergoing medical treatment), or cattle or poultry (e.g., cattle or poultry being used in agriculture). The one or more benefits can comprise at least one of increased mitochondrial energy, treatment or prevention of metabolic fatigue, treatment or prevention of muscle fatigue, improvement in a physiological state linked to metabolic fatigue in one or more cells, improved mobility or improved longevity. Preferably, the NAD+ biosynthesis is increased in one or more cells of the individual, for example one or more cells that are part of at least one body part selected from the group consisting of a liver, a kidney, a brain, and a skeletal muscle. In some embodiments, the composition is administered to an older adult or an elderly individual.

For non-human mammals such as rodents, some embodiments comprise administering an amount of the composition that provides 1.0 mg to 1.0 g of the combination of NARH and NR/kg of body weight of the non-human mammal, preferably 10 mg to 500 mg of combination of NARH and NR/kg of body weight of the non-human mammal, more preferably 25 mg to 400 mg of the combination of NARH and NR/kg of body weight of the mammal, most preferably 50 mg to 300 mg of the combination of NARH and NR/kg of body weight of the non-human mammal.

For humans, some embodiments comprise administering an amount of the composition that provides 1.0 mg to 10.0 g of the combination of NARH and NR/kg of body weight of the human, preferably 10 mg to 5.0 g of the combination of NARH and NR/kg of body weight of the human, more preferably 50 mg to 2.0 g of the combination of NARH and NR/kg of body weight of the human, most preferably 100 mg to 1.0 g of the combination of NARH and NR/kg of body weight of the human.

In some embodiments, at least a portion of the NARH and/or NR is isolated from natural plant sources. Additionally or alternatively, at least a portion of the NARH and/or NR can be chemically synthesized.

As used herein, a “composition consisting essentially of a combination of NARH and NR” contains NARH and NR and does not include, or is substantially free of, or completely free of, any additional compound that affects NAD+ production other than the NARH and NR. In a particular non-limiting embodiment, the composition consists of the combination of NARH and NR and an excipient or one or more excipients.

In some embodiments, the composition consisting essentially of a combination of NARH and NR is optionally substantially free or completely free of other NAD+ precursors.

As used herein, “substantially free” means that any of the other compounds present in the composition is no greater than 1.0 wt. % relative to the amount of the combination of NARH and NR, preferably no greater than 0.1 wt. % relative to the amount of the combination of NARH and NR, more preferably no greater than 0.01 wt. % relative to the amount of the combination of NARH and NR, most preferably no greater than 0.001 wt. % relative to the amount of the combination of NARH and NR.

Another aspect of the present disclosure is a method for increasing intracellular adenine dinucleotide (NAD+) in an individual in need thereof. The method can promote the increase of intracellular levels of NAD+ in cells and tissues for improving cell and tissue survival and overall cell and tissue health. For example, the increase of intracellular levels of NAD+ can provide at least one of increased mitochondrial energy, treatment or prevention of metabolic fatigue, treatment or prevention of muscle fatigue, improvement in a physiological state linked to metabolic fatigue, improved mobility or improved longevity. Preferably, the NAD+ biosynthesis is increased in one or more cells of the individual, for example one or more cells that are part of at least one body part selected from the group consisting of a liver, a kidney, a brain, and a skeletal muscle.

Nicotinamide adenine dinucleotide (NAD+) is considered a coenzyme, and essential cofactor in cellular redox reactions to produce energy. It plays critical roles in energy metabolism, as the oxidation of NADH to NAD+ facilitates hydride-transfer, and consequently ATP generation through mitochondrial oxidative phosphorylation. It also acts as a degradation substrate for multiple enzymes (Canto, C. et al. 2015; Imai, S. et al. 2000; Chambon, P. et al. 1963; Lee, H. C. et al. 1991).

Mammalian organisms can synthesize NAD+ from four different sources. First, NAD+ can be obtained from tryptophan through the 10-step de novo pathway. Secondly, Nicotinic acid (NA) can also be transformed into NAD+ through the 3-step Preiss-Handler path, which converges with the de novo pathway. Thirdly, intracellular NAD+ salvage pathway from nicotinamide (NAM) constitutes the main path by which cells build NAD+, and occurs through a 2-step reaction in which NAM is first transformed into NAM-mononucleotide (NMN) via the catalytic activity of the NAM-phosphoribosyltransferase (NAMPT) and then converted to NAD+ via NMN adenylyltransferase (NMNAT) enzymes. Finally, Nicotinamide Riboside (NR) constitutes yet a fourth path to NAD+, characterized by the initial phosphorylation of NR into NMN by NR kinases (NRKs) (Breganowski, P. et al.; 2004).

Five molecules previously have been known to act as direct extracellular NAD+ precursors: tryptophan, nicotinic acid (NA), nicotinamide (NAM), nicotinic acid riboside (NaR) and nicotinamide riboside (NR). The present invention relates to a combination of NARH and NR, which displays an unexpected synergistic effect in inducing NAD+ when compared to any other combinations of NAD+ precursors. This advantage of the invention supports its therapeutic efficacy.

The method comprises administering an effective amount of a composition comprising a combination of NARH and NR or consisting of NARH and NR to the individual.

Yet another aspect of the present disclosure is a method of treating or preventing (e.g., reducing incidence and/or severity) a mitochondria-related disease or a condition associated with altered mitochondrial function in an individual in need thereof or at risk thereof. The method comprises orally administering to an individual a composition comprising a combination of NaRH and NR in an amount effective to increase NAD+ biosynthesis.

The mitochondria-related disease or condition can be selected from the group consisting of deleterious effects of aging, stress (e.g., oxidative stress), obesity, overweight, reduced metabolic rate, metabolic syndrome, diabetes mellitus, complications from diabetes, hyperlipidemia, neurodegenerative disease, cognitive disorder, stress-induced or stress-related cognitive dysfunction, mood disorder, anxiety disorder, age-related neuronal death or dysfunction, premature aging syndromes (progeria, cockayne syndrome), chronic kidney disease, kidney failure, trauma, infection, cancer, hearing loss, macular degeneration, myopathies and dystrophies, mitochondrial genetic diseases and combinations thereof.

For example, aging is a condition that can be linked to one of the following: oxidative stress, reduced level of glutathione, and lower redox ratio NAD+/NADH. The compositions disclosed herein can treat or prevent these deleterious effects of aging. For example, trigonelline increases NAD+, and the present inventors believe that the increased NAD+ may lead to enhancement of glutathione through redox recycling.

As other examples, depression is linked to low glutathione, and anxiety is linked to oxidative stress. The compositions disclosed herein can treat or prevent these conditions.

These methods can consist essentially of administering the composition comprising a combination of NARH and NR or consisting of a combination of NARH and NR. As used herein, a “method consisting essentially of administering the composition consisting essentially of a combination of NARH and NR” means that any additional compound that affects NAD+ production other than the combination of NARH and NR is not administered within one hour as the administration of the combination of NARH and NR, preferably not administered within two hours as the administration of the combination of NARH and NR, more preferably not administered within three hours as the administration of the combination of NARH and NR, most preferably not administered in the same day as the administration of the combination of NARH and NR. Non-limiting examples of compounds that optionally can be excluded from the method include those disclosed above regarding exclusion from the composition itself.

Yet another aspect of the present disclosure is a method of promoting protective immunity and/or for preventing and/or treating bacterial or viral infections and/or for limiting immune mediated pathology following infection in an individual comprising delivering to the individual in need thereof or at risk thereof, the method comprising orally administering to the individual a composition combination of NARH and NR or consisting of a combination of NARH and NR in an amount effective to increase NAD+ biosynthesis.

Within the context of the present invention, the term “promotion of protective immunity”” means one or more of the following: prevention of infection, anti-pathogen activity, limiting pathogen expansion, promoting pathogen clearance, restriction of pathogen dissemination, recovery from infection, reducing the risk of secondary infection, and/or limiting immune mediated pathology following infection. Promoting protective immunity can be defined by the three levels of immune defence against pathogens (i) mucosal barrier functions of the lung and gastrointestinal tract, (ii) the innate immune response and in particular macrophages with antimicrobial activity and (iii) the adaptive immune response including CD8 T cell activation, which increases anti-viral immunity in the lung.

Within the context of the present invention, the term “infections” includes gastrointestinal infections, respiratory infections (upper and/or lower respiratory tract infections), urinary infections, including both bacterial and viral infections.

Within the context of the present invention, the term gastrointestinal infection means an infection caused by enteropathogens to include but is not limited to Salmonella, Shigella, C. difficile and/or Citrobacter.

Within the context of the present invention, the term “viral infections” means infections caused by viruses, such as for example influenza infection, rotavirus infection and the like. Both innate and adaptive immunity contribute to protective immunity to viral infections.

Within the context of the present invention, the term Respiratory tract infections (RTIs) refers to infectious diseases involving the respiratory tract. An infection of this type usually is further classified as an upper respiratory tract infection (URI or URTI) or a lower respiratory tract infection (LRI or LRTI). Lower respiratory infections, such as pneumonia, tend to be far more severe than upper respiratory infections, such as the common cold. Upper respiratory tract infection (URTI) is an illness caused by an acute infection, which involves the upper respiratory tract, including the nose, sinuses, pharynx, or larynx. This commonly includes nasal obstruction, sore throat, tonsillitis, pharyngitis, laryngitis, sinusitis, otitis media, and the common cold. Most infections are viral in nature, and in other instances, the cause is bacterial. The lower respiratory tract consists of the trachea (windpipe), bronchial tubes, bronchioles, and the lungs. LRIs are bronchitis and pneumonia.

Within the context of the present invention, Pulmonary Diseases and Conditions include:
i) Obstructive lung diseases and conditions, typically affecting (i) the airways and/or (ii) the alveoli.
ii) Lung Airway obstruction diseases and conditions, which affect the trachea, bronchi, and bronchioles which in turn branch to become progressively smaller tubes throughout the lungs. Conditions and diseases that affect the lung airways include, for example: asthma, chronic obstructive pulmonary disease (COPD), chronic bronchitis, emphysema, acute bronchitis and cystic fibrosis.
iii) Lung Alveolar obstruction disease and conditions Alveoli are the air sacs make up most of the lung tissue. Disease and conditions that affect the lung alveoli include, for example, pneumonia, tuberculosis, etc.

It may be appreciated that the compounds, compositions and methods of the present invention may be beneficial to prevent and/or treat bacteria and/or viral infections mentioned above, in particular, to maintain or improve organ tissue function.

Influenza affects both the upper and lower respiratory tracts, but more dangerous strains such as the highly pernicious H5N1 tend to bind to receptors deep in the lungs.

Another aspect of the present disclosure is a unit dosage form of a composition comprising a combination of NARH and NR or consisting of a combination of NARH and NR in an amount effective for treatment or prevention of at least condition selected from the group consisting of deleterious effects of aging, diabetes (type I or type II), complications from diabetes (e.g., diabetic dyslipidemia and/or diabetic microvascular complications such as nephropathy, retinopathy, and/or neuropathy), insulin resistance, metabolic syndrome, dyslipidemia, overweight, obesity, overweight, raised cholesterol levels, raised triglyceride levels, elevated fatty acid levels, fatty liver disease (e.g., non-alcoholic fatty liver disease, including with or without inflammation), cardiovascular disease (e.g., heart failure and/or impaired cardiac contractile function), neurodegenerative disease (e.g., from aging), depression, anxiety, decreased/low motivation, impaired cognitive function, myopathy such as statin-induced myopathy, non-alcoholic steatohepatitis, tinnitus, dizziness, alcohol hangover, hearing impairment, osteoporosis, hypertension, atherosclerosis/coronary artery disease, myocardial damage after stress (e.g., from burns or trauma), traumatic brain injury (including concussions), cystic fibrosis, inflammation, cancer, chemotherapy side effects, HIV infection, stroke, migraine, and brain ischemia.

For example, aging is a condition that can be linked to one of the following: oxidative stress, reduced level of glutathione, and lower redox ratio NAD+/NADH. The compositions disclosed herein can treat or prevent these deleterious effects of aging. For example, the combination of NARH and NR increases NAD+, and the present inventors believe that the increased NAD+ may lead to enhancement of glutathione through redox recycling.

As other examples, depression is linked to low glutathione, and anxiety is linked to oxidative stress. The compositions disclosed herein can treat or prevent these conditions.

Another aspect of the present disclosure is a method of treating at least one of these conditions, the method comprising administering a therapeutically effective amount of a composition comprising a combination of NARH and NR or consisting of a combination of NARH and NR to the individual having the condition. Another aspect of the present disclosure is a method of preventing at least one of these conditions, the method comprising administering a prophylactically effective amount of a composition comprising a combination of NARH and NR or consisting combination of NARH and NR to an individual at risk of the at least one condition.

The composition comprising a combination of NARH and NR or consisting of a combination of NARH and NR can treat or prevent an eye condition resulting directly or indirectly from low GSH levels, including low levels in the lens of the eye that is known for being rich in glutathione. Non-limiting examples of such conditions include cataracts and/or glaucoma, presbyopia (loss of near vision with aging requiring reading glasses), and presbyacusis (loss of hearing with aging, which requires a hearing aid).

Yet another aspect of the present disclosure is a method of delaying off-set of metabolic decline, decreasing oxidative stress, maintaining immune function and/or maintaining cognitive function in a healthy older adult. The method comprises administering an effective amount of a composition comprising a combination of NARH and NR or consisting of a combination of NARH and NR to the healthy older adult.

Yet another aspect of the present disclosure is a unit dosage form of a composition comprising a combination of NARH and NR or consisting of a combination of NARH and NR in an amount effective for weight management. “Weight management” for an adult (e.g., at least eighteen years from birth) means that the individual has approximately the same body mass index (BMI) after one week of consumption of the composition, preferably after one month of consumption of the composition, more preferably after one year of consumption of the composition, relative to their BMI when consumption of the composition was initiated. “Weight management” for younger individuals means that the BMI is approximately the same percentile relative to an individual of a corresponding age after one week of consumption of the composition, preferably after one month of consumption of the composition, more preferably after one year of consumption of the composition, relative to their BMI percentile when consumption of the composition was initiated.

In another aspect, the present disclosure provides a method of improving cognitive function. The method comprises administering an effective amount of a composition comprising a combination of NARH and NR or consisting of a combination of NARH and NR to an individual. The cognitive function can be selected from the group consisting of perception, memory, attention, speech comprehension, speech generation, reading comprehension, creation of imagery, learning, reasoning, and combinations thereof. In an embodiment, the individual does not have a cognitive disorder; alternatively, the individual has a cognitive disorder. The individual can be elderly and/or can have cognitive decline associated with aging.

Yet another aspect of the present disclosure is a method of improving one or more of fetal metabolic programming for prevention of later development of obesity, overweight and/or diabetes, maternal and fetal health in gestational diabetes, exercise capacity and physical function, quality of life, longevity, memory, cognition, post-traumatic recovery and survival (e.g., post-surgical, post-sepsis, post-blunt or penetrating trauma due to accident or physical assault), or recovery from trauma and surgery. The method comprises administering an effective amount of composition comprising a combination of NARH and NR or consisting of a combination of NARH and NR to an individual at risk thereof or in need thereof.

Yet another aspect of the present disclosure is a method of (i) treating or preventing at least one physical state selected from the group consisting of oxidative stress, a condition associated with oxidative stress (e.g., aging and its effects such as skin aging), a reduced level of glutathione, a condition associated with a reduced level of glutathione, or (ii) improving one or more of fetal metabolic programming for prevention of later development of obesity, overweight, pre-diabetes and/or diabetes, maternal and fetal health in gestational diabetes, exercise capacity and physical function, quality of life, longevity, memory, cognition, post-traumatic recovery and survival, or recovery from trauma and surgery. The method comprises administering an effective amount of a composition comprising a combination of NARH and NR or consisting of a combination of NARH and NR to an individual at risk thereof or in need thereof. For example, the increased NAD+ may lead to enhancement of glutathione through redox recycling.

In biology and psychology, the term “stress” refers to the consequence of the failure of a human or other animal to respond appropriately to physiological, emotional, or physical threats, whether actual or imagined. The psychobiological features of stress may present as manifestations of oxidative stress, i.e., an imbalance between the production and manifestation of reactive oxygen species and the ability of a biological system readily to detoxify the reactive intermediates or to repair the resulting damage. Disturbances in the normal redox state of tissues can cause toxic effects through the production of peroxides and free radicals that damage all of the components of the cell, including proteins, lipids, and DNA. Some reactive oxidative species can even act as messengers through a phenomenon called “redox signaling.”

In humans, oxidative stress is involved in many diseases. Examples include atherosclerosis, Parkinson's disease, heart failure, myocardial infarction, Alzheimer's disease, schizophrenia, bipolar disorder, fragile X syndrome, and chronic fatigue syndrome.

One source of reactive oxygen under normal conditions in humans is the leakage of activated oxygen from mitochondria during oxidative phosphorylation. Other enzymes capable of producing superoxide (O2-) are xanthine oxidase, NADPH oxidases and cytochromes P450. Hydrogen peroxide, another strong oxidizing agent, is produced by a wide variety of enzymes including several oxidases. Reactive oxygen species play important roles in cell signaling, a process termed redox signaling. Thus, to maintain proper cellular homeostasis a balance must be struck between reactive oxygen production and consumption.

Oxidative stress contributes to tissue injury following irradiation and hyperoxia. It is also suspected to be important in neurodegenerative diseases, including Alzheimer's disease, Parkinson's disease, amyotrophic lateral sclerosis (ALS), and Huntington's disease.

Oxidative stress is also thought to be linked to certain cardiovascular diseases, since oxidation of low-density lipoprotein (LDL) in the vascular endothelium is a precursor to plaque formation. Oxidative stress also plays a role in the ischemic cascade due to oxygen reperfusion injury following hypoxia. This cascade includes both strokes and heart attacks. Oxidative stress has also been implicated in chronic fatigue syndrome.

Moreover, the free radical theory of aging suggests that the biological process of aging results in increased oxidative stress in elderly humans. The ability of a cell to resist the damaging potential of oxidative stress is determined by a vital balance between generation of oxidant free radicals and the defensive array of antioxidants available to the cell. There are multiple antioxidant defense systems and of these, glutathione (GSH) is the most abundant intracellular component of overall antioxidant defenses. GSH, a tripeptide, is synthesized from precursor amino-acids glutamate, cysteine, and glycine in two steps catalyzed by glutamate cysteine ligase (GCL, also known as gamma-glutamylcysteine synthetase, EC 6.3.2.2) and gamma-L-glutamyl-L-cysteine:glycine ligase (also known as glutathione synthetase, EC 6.3.2.3), and GSH synthesis occurs de novo in cells.

In each of the compositions and methods disclosed herein, the composition is preferably a food product or beverage product, including food additives, food ingredients, functional foods, dietary supplements, medical foods, nutraceuticals, oral nutritional supplements (ONS) or food supplements.

The composition can be administered at least one day per week, preferably at least two days per week, more preferably at least three or four days per week (e.g., every other day), most preferably at least five days per week, six days per week, or seven days per week. The time period of administration can be at least one week, preferably at least one month, more preferably at least two months, most preferably at least three months, for example at least four months. In some embodiments, dosing is at least daily; for example, a subject may receive one or more doses daily, in an embodiment a plurality of doses per day. The combination can be administered in a single dose per day or in multiple separate doses per day. In some embodiments, the administration continues for the remaining life of the individual. In other embodiments, the administration occurs until no detectable symptoms of the medical condition remain. In specific embodiments, the administration occurs until a detectable improvement of at least one symptom occurs and, in further cases, continues to remain ameliorated.

The compositions disclosed herein may be administered to the subject enterally, e.g., orally, or parenterally. Non-limiting examples of parenteral administration include intravenously, intramuscularly, intraperitoneally, subcutaneously, intraarticularly, intrasynovially, intraocularly, intrathecally, topically, and inhalation. As such, non-limiting examples of the form of the composition include natural foods, processed foods, natural juices, concentrates and extracts, injectable solutions, microcapsules, nano-capsules, liposomes, plasters, inhalation forms, nose sprays, nosedrops, eyedrops, sublingual tablets, and sustained-release preparations.

The compositions disclosed herein can use any of a variety of formulations for therapeutic administration. More particularly, pharmaceutical compositions can comprise appropriate pharmaceutically acceptable carriers or diluents and may be formulated into preparations in solid, semi-solid, liquid or gaseous forms, such as tablets, capsules, powders, granules, ointments, solutions, suppositories, injections, inhalants, gels, microspheres, and aerosols. As such, administration of the composition can be achieved in various ways, including oral, buccal, rectal, parenteral, intraperitoneal, intradermal, transdermal, and intratracheal administration. The active agent may be systemic after administration or may be localized by the use of regional administration, intramural administration, or use of an implant that acts to retain the active dose at the site of implantation.

In pharmaceutical dosage forms, the compounds may be administered as their pharmaceutically acceptable salts. They may also be used in appropriate association with other pharmaceutically active compounds. The following methods and excipients are merely exemplary and are in no way limiting.

For oral preparations, the compounds can be used alone or in combination with appropriate additives to make tablets, powders, granules or capsules, for example, with conventional additives, such as lactose, mannitol, corn starch or potato starch; with binders, such as crystalline cellulose, cellulose functional derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The compounds can be formulated into preparations for injections by dissolving, suspending or emulsifying them in an aqueous or non-aqueous solvent, such as vegetable or other similar oils, synthetic aliphatic acid glycerides, esters of higher aliphatic acids or propylene glycol; and if desired, with conventional, additives such as solubilizers, isotonic agents, suspending agents, emulsifying agents, stabilizers and preservatives.

The compounds can be utilized in an aerosol formulation to be administered by inhalation. For example, the compounds can be formulated into pressurized acceptable propellants such as dichlorodifluoromethane, propane, nitrogen and the like.

Furthermore, the compounds can be made into suppositories by mixing with a variety of bases such as emulsifying bases or water-soluble bases. The compounds can be administered rectally by a suppository. The suppository can include a vehicle such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition. Similarly, unit dosage forms for injection or intravenous administration may comprise the compounds in a composition as a solution in sterile water, normal saline or another pharmaceutically acceptable carrier, wherein each dosage unit, for example, mL or L, contains a predetermined amount of the composition containing one or more of the compounds.

Compositions intended for a non-human animal include food compositions to supply the necessary dietary requirements for an animal, animal treats (e.g., biscuits), and/or dietary supplements. The compositions may be a dry composition (e.g., kibble), semi-moist composition, wet composition, or any mixture thereof. In one embodiment, the composition is a dietary supplement such as a gravy, drinking water, beverage, yogurt, powder, granule, paste, suspension, chew, morsel, treat, snack, pellet, pill, capsule, tablet, or any other suitable delivery form. The dietary supplement can comprise a high concentration of the UFA and NORC, and B vitamins and antioxidants. This permits the supplement to be administered to the animal in small amounts, or in the alternative, can be diluted before administration to an animal. The dietary supplement may require admixing, or can be admixed with water or other diluent prior to administration to the animal.

“Pet food” or “pet treat compositions” comprise from about 15% to about 50% crude protein. The crude protein material may comprise vegetable proteins such as soybean meal, soy protein concentrate, corn gluten meal, wheat gluten, cottonseed, and peanut meal, or animal proteins such as casein, albumin, and meat protein. Examples of meat protein useful herein include pork, lamb, equine, poultry, fish, and mixtures thereof. The compositions may further comprise from about 5% to about 40% fat. The compositions may further comprise a source of carbohydrate. The compositions may comprise from about 15% to about 60% carbohydrate. Examples of such carbohydrates include grains or cereals such as rice, corn, milo, sorghum, alfalfa, barley, soybeans, canola, oats, wheat, and mixtures thereof. The compositions may also optionally comprise other materials such as dried whey and other dairy by-products.

In some embodiments, the ash content of the pet food composition ranges from less than 1% to about 15%, and in one aspect, from about 5% to about 10%.

The moisture content can vary depending on the nature of the pet food composition. In a one embodiment, the composition can be a complete and nutritionally balanced pet food. In this embodiment, the pet food may be a “wet food”, “dry food”, or food of intermediate moisture content. “Wet food” describes pet food that is typically sold in cans or foil bags, and has a moisture content typically in the range of about 70% to about 90%. “Dry food” describes pet food which is of a similar composition to wet food, but contains a limited moisture content, typically in the range of about 5% to about 15% or 20%, and therefore is presented, for example, as small biscuit-like kibbles. In one embodiment, the compositions have moisture content from about 5% to about 20%. Dry food products include a variety of foods of various moisture contents, such that they are relatively shelf-stable and resistant to microbial or fungal deterioration or contamination. Also included are dry food compositions which are extruded food products, such as pet foods, or snack foods for companion animals.

### REFERENCES

Canto, C., K. J. Menzies, and J. Auwerx, 2015. NAD(+) Metabolism and the Control of Energy Homeostasis: A Balancing Act between Mitochondria and the Nucleus. Cell Metab. 22(1): 31-53.
Chambon, P., J. D. Weill, and P. Mandel, 1963. Nicotinamide mononucleotide activation of new DNA-dependent polyadenylic acid synthesizing nuclear enzyme. Biochem Biophys Res Commun. 1139-43.
Imai, S., C. M. Armstrong, M. Kaeberlein, and L. Guarente, 2000. Transcriptional silencing and longevity protein Sir2 is an NAD-dependent histone deacetylase. Nature. 403(6771): 795-800.
Lee, H. C. and R. Aarhus, 1991. ADP-ribosyl cyclase: an enzyme that cyclizes NAD+ into a calcium-mobilizing metabolite. Cell Regul. 2(3): 203-9.

### Examples

The following non-limiting examples present scientific data developing and supporting the concept of a composition comprising a combination of NARH and NR for cellular nutrition.

### Example 1. Comparative Example in Mouse Hepatocytes

AML12 cells were treated with:
i) dihydronicotinic acid riboside (NARH) and nicotinamide riboside (NR), alone or in combination for 2 hours. NARH and NR were used at the concentration of 0.5 mM.
ii) dihydronicotinic acid riboside (NARH) and nicotinic acid (NA), alone or in combination for 2 hours. NARH and NA were used at the concentration of 0.5 mM.
iii) dihydronicotinic acid riboside (NARH) and nicotinamide (NAM) alone or in combination for 2 hours. NARH and NAM were used at the concentration of 0.5 and 5 mM, respectively.
iv) dihydronicotinic acid riboside (NARH) and nicotinic acid riboside (NAR) alone or in combination for 2 hours. NARH and NAR were used at the concentration of 0.5 mM.
v) dihydronicotinic acid riboside (NARH) and dihydronicotinamide riboside (NRH) alone or in combination for 2 hours. NARH and NRH were used at the concentration of 0.5 and 0.01 mM, respectively.
vi) nicotinamide riboside (NR) and nicotinic acid (NA) alone or in combination for 2 hours. NR and NA were used at the concentration of 0.5 mM.
vii) nicotinamide riboside (NR) and nicotinamide (NAM) alone or in combination for 2 hours. NR and NAM were used at the concentration of 0.5 and 5 mM, respectively
viii) nicotinamide riboside (NR) and nicotinic acid riboside (NAR) alone or in combination for 2 hours. NR and NAR were used at the concentration of 0.5 mM
ix) nicotinic acid (NA) and nicotinamide (NAM) alone or in combination for 2 hours. NA and NAM were used at the concentration of 0.5 and 5 mM
x) nicotinic acid (NA) and nicotinic acid riboside (NAR) alone or in combination for 2 hours. NA and NAR were used at the concentration of 0.5 mM.
xi) nicotinamide (NAM), nicotinic acid riboside (NAR) alone or in combination for 2 hours. NAM and NAR were used at the concentration of 5 and 0.5 mM
xii) nicotinic acid riboside (NAR), dihydronicotinamide riboside (NRH) alone or in combination for 2 hours. NAR and NRH were used at the concentration of 0.5 and 0.01 mM, respectively.

After treatment, cells were trypsinized and collected for NAD+ and protein content measurements. NAD+ was extracted and quantified using an EnzyChrom NAD/NADH Assay Kit (BioAssay Systems; #E2ND-100) according to the manufacturer's instructions. Proteins were quantified with BCA Protein Assay Kit (Pierce™; #23225).

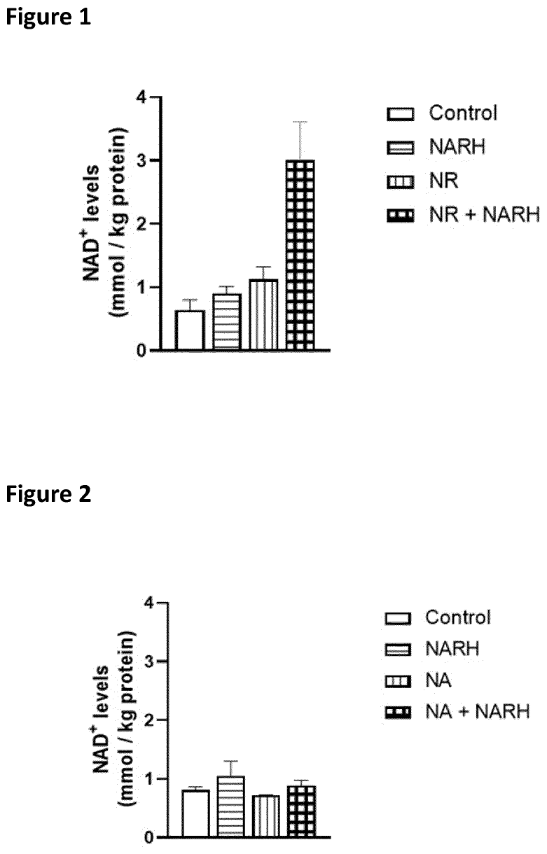

Results are presented in FIGS. 1 to 12.

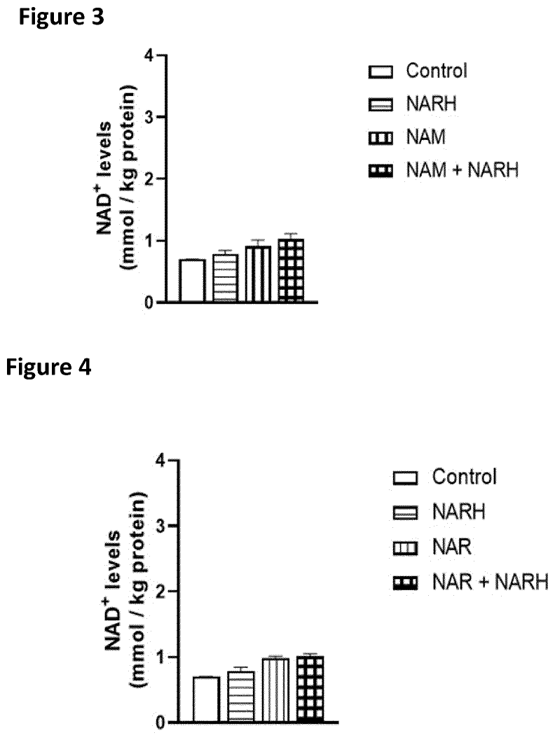

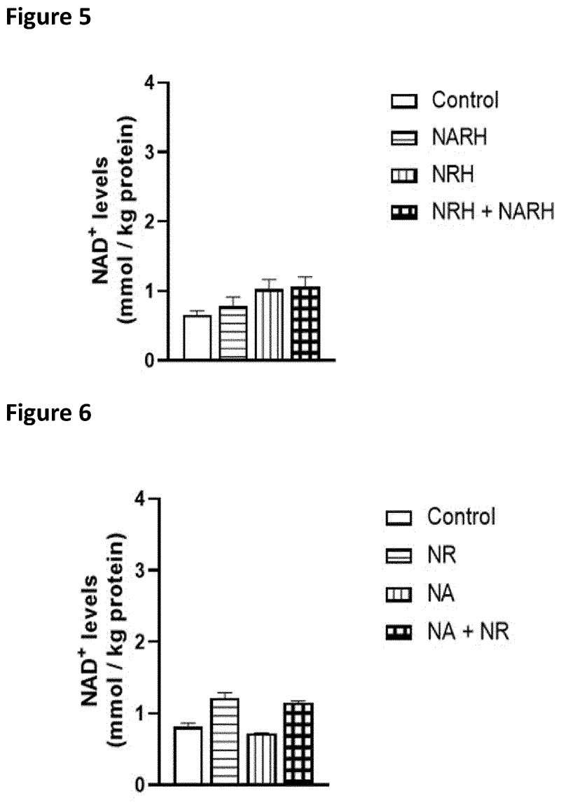

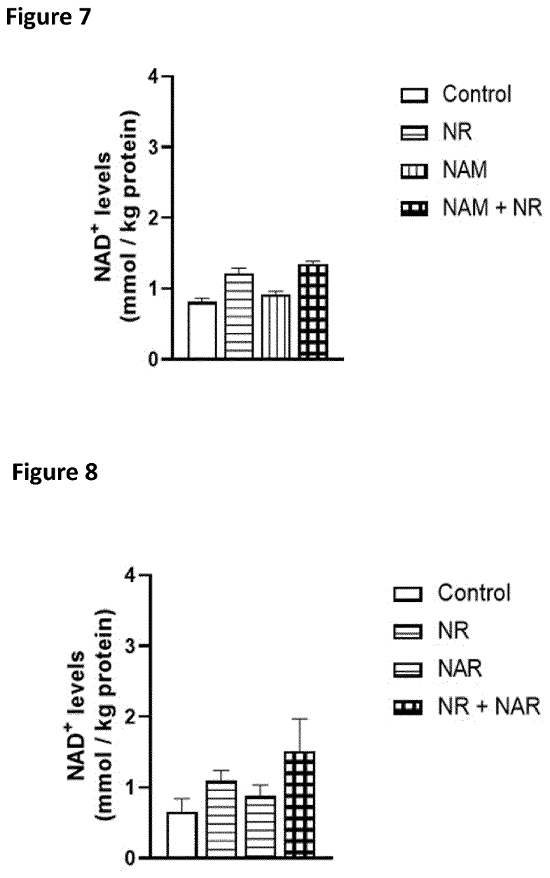

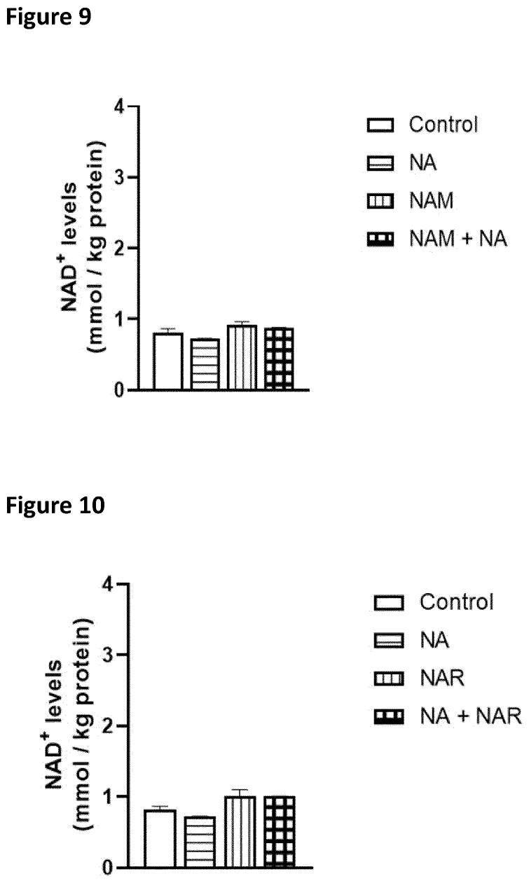

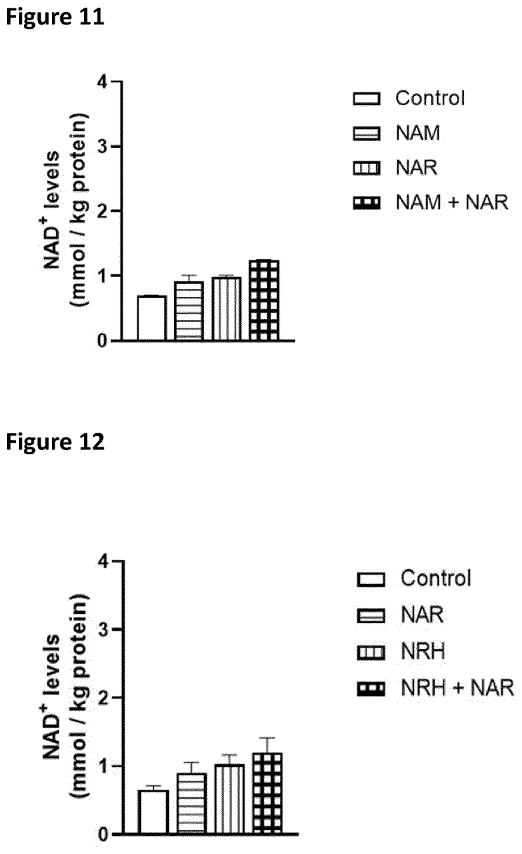

FIG. 1 shows that the combination of NARH and NR synergistically induces NAD+ in mouse hepatocytes, whereas combinations of other NAD+ precursors do not exhibit synergism in NAD+ induction: e.g. NARH and NA (FIG. 2), NARH and NAM (FIG. 3), NARH and NAR (FIG. 4), NARH and NRH (FIG. 5), NR and NA (FIG. 6), NR and NAM (FIG. 7), NR and NAR (FIG. 8), NA and NAM (FIG. 9), NA and NAR (FIG. 10), NAM and NAR (FIG. 11) or NAR and NRH (FIG. 12).

### Example 2: NARH and NR Combination Synergistically Induces NAD+ in Rat Pancreatic p Cells

INS-1 cells were treated with dihydronicotinic acid riboside (NARH) and nicotinamide riboside (NR), alone or in combination for 2 hours. NARH and NR were used at the concentration of 0.5 mM. After treatment, cells were trypsinized and collected for NAD+ and protein content measurements. NAD+ was extracted and quantified using an EnzyChrom NAD/NADH Assay Kit (BioAssay Systems; #E2ND-100) according to the manufacturer's instructions. Proteins were quantified with BCA Protein Assay Kit (Pierce™; #23225).

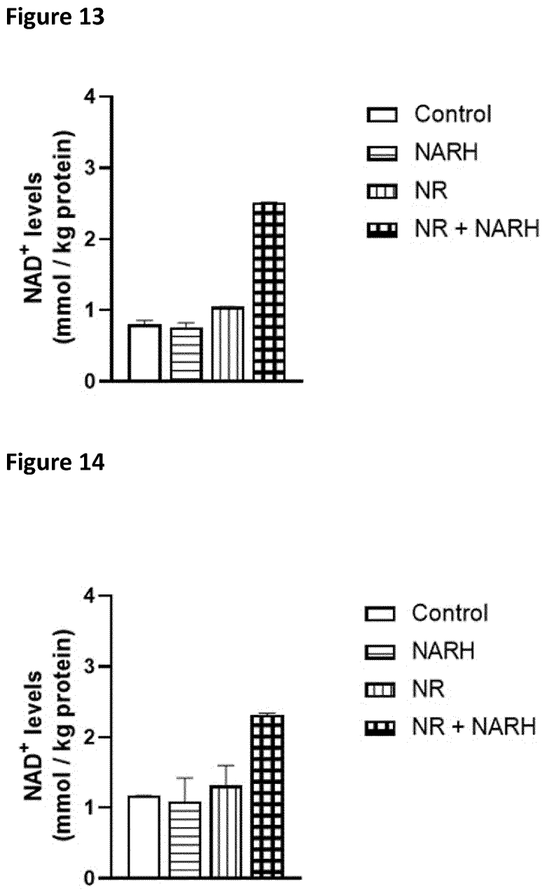

Results are presented in FIG. 13. This shows that NARH and NR combination synergistically induces NAD+ in rat pancreatic p cells.

### Example 3: NARH and NR Combination, but not NARH or NR Alone, Induces NAD+ in Mouse Fibroblast

MEF cells were treated with dihydronicotinic acid riboside (NARH) and nicotinamide riboside (NR), alone or in combination for 2 hours. NARH and NR were used at the concentration of 0.5 mM. After treatment, cells were trypsinized and collected for NAD+ and protein content measurements. NAD+ was extracted and quantified using an EnzyChrom NAD/NADH Assay Kit (BioAssay Systems; #E2ND-100) according to the manufacturer's instructions. Proteins were quantified with BCA Protein Assay Kit (Pierce™; #23225).

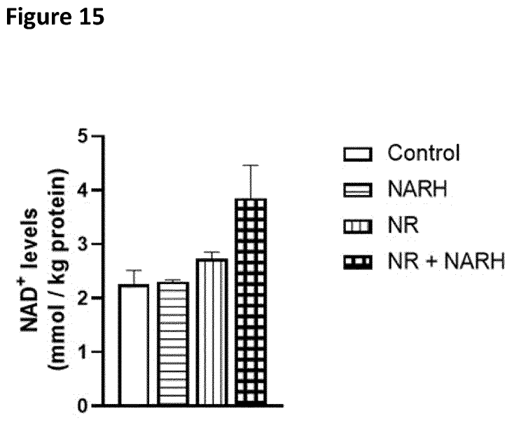

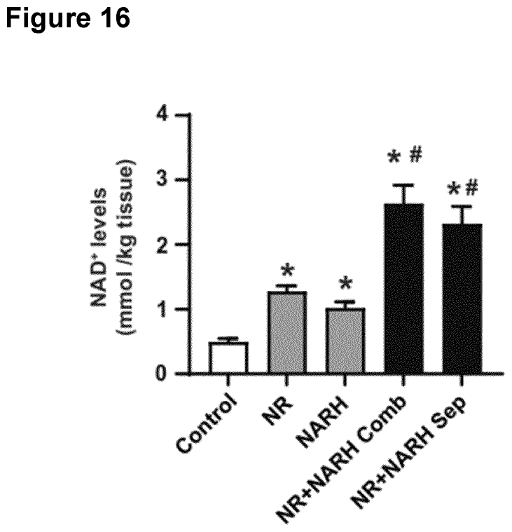

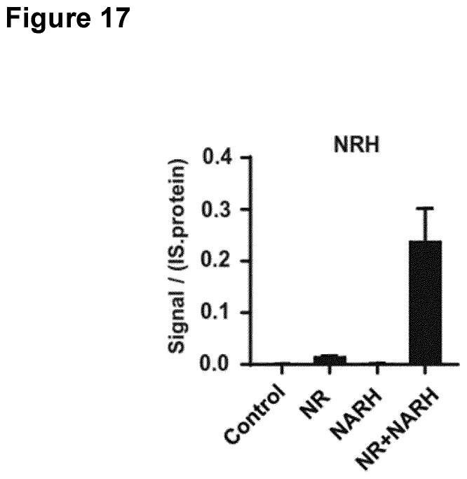

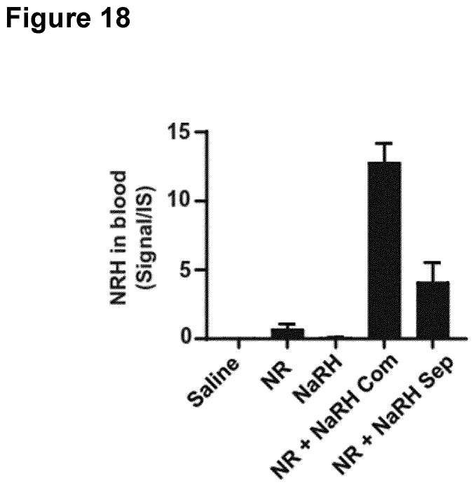

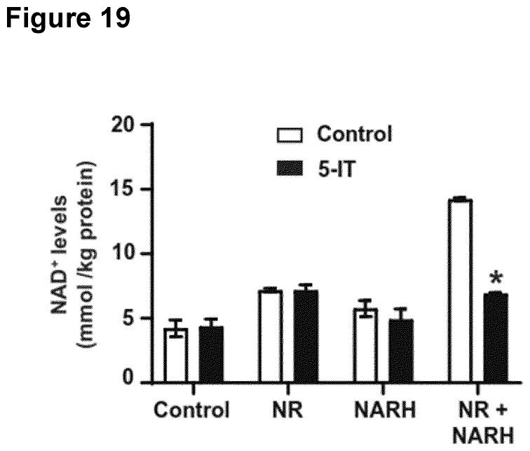

Results are presented in FIG. 14. This shows that NARH and NR combination, but not NARH or NR alone, induces NAD+ in mouse fibroblast.

### Example 4. NARH and NR Combination, but not NARH or NR Alone, Induces NAD+ in Mouse Macrophages

RAW 264.7 cells were treated with dihydronicotinic acid riboside (NARH) and nicotinamide riboside (NR), alone or in combination for 2 hours. NARH and NR were used at the concentration of 0.5 mM. After treatment, cells were scraped and collected for NAD+ and protein content measurements. NAD+ was extracted and quantified using an EnzyChrom NAD/NADH Assay Kit (BioAssay Systems; #E2ND-100) according to the manufacturer's instructions. Proteins were quantified with BCA Protein Assay Kit (Pierce™; #23225).

Results are presented in FIG. 15. This shows that NARH and NR combination, but not NARH or NR alone, induces NAD+ in mouse macrophages.

### Example 5. NR and NARH have Synergistic Effects on Hepatic NAD⁺ Levels

After a 2 hour fast, mice (n=5 per group) were intraperitoneally injected with saline (as vehicle) or 500 mg/kg of NR, NARH or NR+NARH. For the NR+NARH combination, in one group both compounds were injected using a single mixture with both compounds (NR+NARH Comb) and in the second group the compounds were injected separately, one in the left side and one in the right side of the peritoneum (NR+NARH Sep). One hour later, liver tissue was snap frozen and NAD⁺ levels were measured. All data is presented as mean+/−SEM for n=4-5 mice per group. * indicates p<0.05 vs. vehicle treated group. # indicates p<0.05 vs. single compound treatments.

Results are presented in FIG. 16. This shows that NARH and NR combination, have synergistic effects on hepatic NAD+ levels.

### Example 6. The Combination of NR+NARH Leads to Increased Intracellular Content of NRH in AML12 Hepatocytes

AML12 cells were treated with either PBS (as control), NR (0.5 mM), NARH (0.5 mM) or both. Then, 2 hours later, cells were flash-frozen and processed to evaluate intracellular levels through LC-MS methods (see Giner et al., 2021; doi: 10.3390/ijms221910598). All data is presented as mean+/−SEM of n=3 experiments.

Results are presented in FIG. 17. This shows that the combination of NR+NARH leads to increased intracellular content of NRH in AML12 hepatocytes.

### Example 7. NR and NARH Lead to Increased NRH Levels in Circulation

After a 2 hour fast, mice (n=5 per group) were intraperitoneally injected with saline (as vehicle) or 500 mg/kg of NR, NARH or NR+NARH. For the NR+NARH combination, in one group both compounds were injected using a single mixture with both compounds (NR+NARH Comb) and a second group where the compounds were injected separately, one in the left and one in the right side of the peritoneum (NR+NARH Sep). One hour later, blood was collected and NRH levels were measured by LC-MS (see Giner et al., 2021; doi: 10.3390/ijms221910598). All data is presented as mean+/−SEM of n=3 experiments.

Results are presented in FIG. 18. This shows that NR and NARH lead to increased NRH levels in circulation.

### Example 8. NR and NARH Require the NRH Path for NAD⁺ Synthesis, Characterized by Adenosine Kinase Activity

AML12 cells were treated with DMSO (as vehicle) or the adenosine kinase inhibitor 5-IT (1 μM;) for 1 hour and then treated with either PBS (as control), NR (0.5 mM), NARH (0.5 mM) or both. Two hours later, acidic extracts were obtained to evaluate NAD⁺ levels. All data is presented as mean+/−SEM of n=3 experiments. * indicates p<0.05 vs. the respective vehicle treated group.

Results are presented in FIG. 19. This shows that NR and NARH require the NRH path for NAD+ synthesis, characterized by adenosine kinase activity.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method for increasing intracellular nicotinamide adenine dinucleotide (NAD+) in an individual, the method comprising orally administering to the subject a composition comprising a combination of NARH and NR in an amount effective to increase NAD+ biosynthesis in one or more cells of the individual.

2. Method according to claim 1, wherein the composition is administered enterally.

3. A Method according to claim 1, wherein the composition is selected from the group consisting of a food product, a food supplement, an oral nutritional supplement (ONS), a medical food, and combinations thereof.

4. Method according to claim 1, wherein at least a portion of the one or more cells are part of at least one body part selected from the group consisting of a liver, a kidney, a brain, and a skeletal muscle.

5. A unit dosage form of a composition consisting of a combination of NARH and NR, in an amount effective to increase NAD+ biosynthesis in an individual.

6. The unit dosage form of claim 5, which is selected from the group consisting of a food product, a food supplement, an oral nutritional supplement (ONS), a medical food, and combinations thereof.

7. The unit dosage form of claim 5, wherein the composition is formulated for enteral administration.

8. A method of achieving at least one result selected from the group consisting of (i) increased mitochondrial energy in one or more cells, (ii) improvement in a physiological state linked to metabolic fatigue in one or more cells, (iii) treatment or prevention of metabolic fatigue in one or more cells, (iv) treatment or prevention of muscle fatigue, (v) improved mobility and (vi) improved longevity, the method comprising orally administering to an individual a composition combination of NARH and NR in an amount effective to increase NAD+ biosynthesis.

9-10. (canceled)

11. The method according to claim 8, wherein the NAD+ biosynthesis is increased in one or more cells that are part of at least one body part selected from the group consisting of a liver, a kidney, a brain, and a skeletal muscle.

12-13. (canceled)

14. The method according to claim 8, wherein the composition is administered enterally.

15. The method according to claim 8, wherein the composition is selected from the group consisting of a food product, a food supplement, an oral nutritional supplement (ONS), a medical food, and combinations thereof.

16-18. (canceled)
